(19) United States
(12) Patent Application Publication
(10) Pub. No.: US 2014/0309672 A1
(43) Pub. Date: Oct. 16, 2014

(54) CLOT BUSTER ASPIRATION CATHETER

(71) Applicant: Cook Medical Technologies LLC, Bloomington, IN (US)

(72) Inventors: Nicos Labropoulos, Coran, NY (US); Antonios Gasparis, Miller Place, NY (US); Frank Fischer, Bloomington, IN (US); Kurt Tekulve, Ellettsville, IN (US)

(73) Assignee: Cook Medical Technologies LLC, Bloomington, IN (US)

(21) Appl. No.: 14/211,295

(22) Filed: Mar. 14, 2014

Related U.S. Application Data
(60) Provisional application No. 61/794,423, filed on Mar. 15, 2013.

(51) Int. Cl. A61B 17/3207; A61M 1/00

(52) U.S. Cl. CPC A61B 17/320725; A61M 1/008
USPC 606/159

(57) ABSTRACT

A medical device for breaking up and aspirating material, especially intravenous clots, having a catheter, a barrel, and a sheath is disclosed. The catheter has a lumen through which material is aspirated. The barrel has a first and second end connected by a plurality of blades that are self-biased to extend outward. The sheath provides a selective radial constraint such that the blades are constrained when the sheath covers the barrel and unconstrained when the sheath is retracted.

### RELATED APPLICATIONS

The present patent document claims the benefit of the filing date under 35 U.S.C. §119(e) of Provisional U.S. Patent Application Ser. No. 61/794,423, filed Mar. 15, 2013, which is hereby incorporated by reference.

### FIELD

Embodiments of the present invention relate to methods and apparatuses for breaking up clots in blood vessels and collecting the broken up clot, and more particularly to a catheter designed to break up and collect a clot.

### BACKGROUND

Deep vein thrombosis (DVT) is a condition in which blood clots form in the large veins of the lower portions of the body. If a blood clot breaks free, it may travel to other parts of the body and cause significant damage. For instance, if a blood clot were to travel to the heart and lungs through the inferior vena cava a pulmonary embolization could result. A pulmonary embolization may be fatal if the clot interferes with the pumping of blood by the heart. If the clot passes through the heart, it may lodge in the pulmonary arteries inhibiting the oxygenation of blood.

Current methods of treating DVT include administering Heparin, an anticoagulant, to prevent further clots from forming and performing intravenous procedures using an aspiration catheter to collect the clot. In some instances a clot busting device may be used in combination with the aspiration catheter to break up the clot into smaller pieces that can be aspirated. The clot busting device is typically delivered through a lumen of the aspiration catheter and occupies at least a portion of the cross sectional area of the lumen. Because the clot buster reduces the cross sectional area of the lumen, the clots must be broken up to a smaller size than when an aspiration catheter is used alone, or the clot buster must be removed from the lumen to aspirate larger clot pieces. However, it is dangerous to break up the clot without simultaneous aspirating the clot pieces, as the clot pieces may travel before being aspirated, causing the very event the surgery is designed to prevent.

It would be beneficial to have a device and methods for breaking up a clot while simultaneously aspirating the clot pieces without reducing the efficacy of the aspiration catheter.

### SUMMARY

Embodiments of the invention include a catheter assembly comprising a barrel, an elongated tubular body, and a sheath. The barrel is formed from a shape memory material and has a first end with an outside diameter, and a second end spaced apart from the first end. A longitudinal axis extends from the first end to the second end and a plurality of longitudinal blades extend proximate the first end to proximate the second end. A film is disposed on a portion of the plurality of blades. The barrel is biased to an expanded state in which a portion of each of the longitudinal blades extends beyond the outside diameter of the first end. The elongated tubular body has a lumen disposed therein and a distal end is coupled to the first end of the barrel. The sheath is disposed about the barrel and is adapted to translate axially from a first position about the barrel to a second position about the elongated tubular body. At the first position the sheath constrains the barrel in a compacted configuration in which the plurality of longitudinal blades does not extend beyond the first outside diameter.

### BRIEF DESCRIPTION OF THE DRAWINGS

To further clarify the above and other advantages and features of the one or more present inventions, reference to specific embodiments thereof are illustrated in the appended drawings. The drawings depict only typical embodiments and are therefore not to be considered limiting. One or more embodiments will be described and explained with additional specificity and detail through the use of the accompanying drawings in which:

FIG. 1 depicts a distal end of an embodiment of a clot buster catheter.

FIG. 1b depicts a distal end of another embodiment of a clot buster catheter.

FIG. 2 depicts a clot buster in a compacted configuration.

FIG. 3 depicts an alternative embodiment of a clot buster.

FIG. 4 depicts an alternative embodiment of a clot buster.

FIG. 5 depicts an alternative embodiment of a clot buster.

FIG. 6 depicts a blood vessel having a clot.

FIG. 7 depicts the blood vessel of FIG. 6 having a clot buster catheter having a clot buster in a compacted configuration proximate the clot.

FIG. 8 depicts the blood vessel of FIG. 6 having a clot buster catheter with an expanded clot buster proximate the clot.

FIG. 9 depicts the blood vessel of FIG. 6 having a clot buster breaking up the clot.

The drawings are not necessarily to scale.

### DETAILED DESCRIPTION

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together.

Various embodiments of the present inventions are set forth in the attached figures and in the Detailed Description as provided herein and as embodied by the claims. It should be understood, however, that this Detailed Description does not contain all of the aspects and embodiments of the one or more present inventions, is not meant to be limiting or restrictive in any manner, and that the invention(s) as disclosed herein is/are and will be understood by those of ordinary skill in the art to encompass obvious improvements and modifications thereto.

Additional advantages of the present invention will become readily apparent from the following discussion, particularly when taken together with the accompanying drawings.

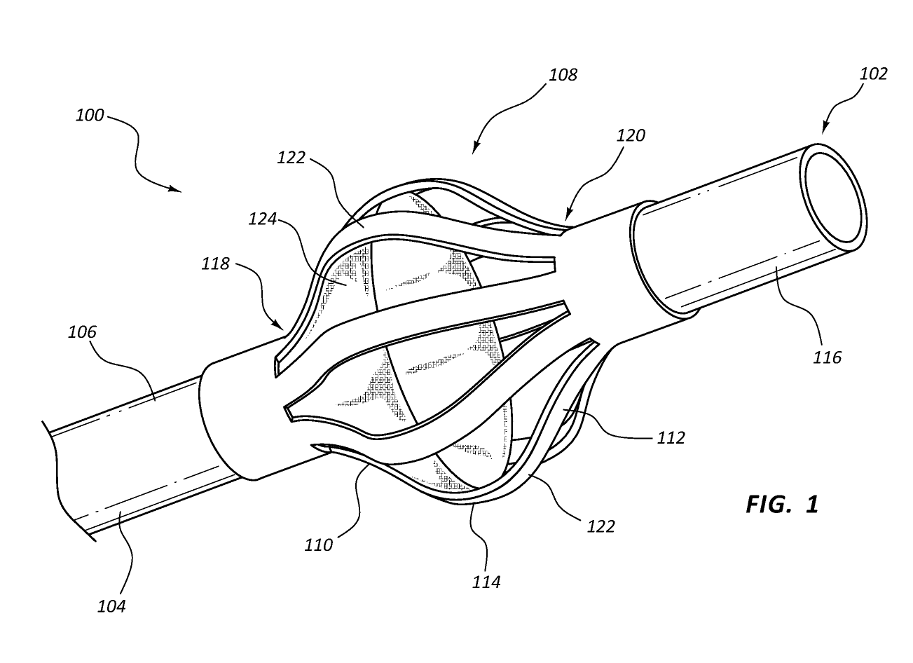

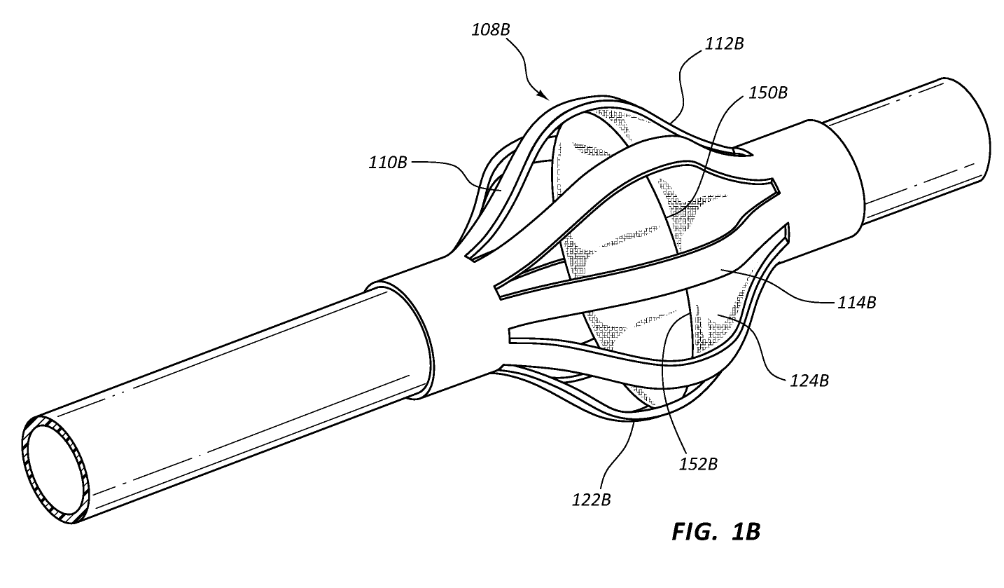

FIG. 1 illustrates a distal end 102 of an embodiment of a catheter assembly 100 adapted for clot busting and clot aspiration. The catheter assembly 100 has a clot buster 108 disposed proximate the distal end 102, and a tubular body 106 extending from a proximal end (not shown) to the clot buster 108. A lumen 104 extends from the proximal end to the clot buster 108 and provides fluid communication between the proximal end and the clot buster 108. A tip 116 is disposed at the distal end 102 of the catheter assembly 100 and may be formed of a soft material, similar to a traditional catheter tip.

The tubular body 106 is of construction similar to a traditional sheath or catheter tubing. The tubular body 106 may be formed of a material and have a wall thickness sufficient to withstand a vacuum applied to the lumen 104 and sufficient to transmit rotational and axial force from the proximal end of the tubular body 106 to the clot buster 108.

The clot buster 108 has a double conical shape with a first cone 110 and a second cone 112 joined at a base 114. The base 114 is typically located at a point midway between a first cone apex 118 and a second cone apex 120. The tubular body 106 is joined to the clot buster 108 near the first cone apex 118 and the tip 116 is joined to the clot buster 108 near the second cone apex. As shown in FIG. 1, the cones 110, 112 need not be precise cones and may only generally resemble cones. The clot buster 108 may be formed out of a single piece of material that has been shaped to the double cone shape, or it may be formed of a plurality of pieces joined together to form the double cone shape.

The clot buster 108 is ideally formed out of a rigid material that has shape memory. For example, the clot buster 108 may be formed of Nitinol tubing, but other materials are possible, such as thermoplastics. The clot buster 108 of FIG. 1 is shown in an expanded form, in which a plurality of struts 122 extends radially outward from the axis of the catheter assembly 100. FIG. 2 illustrates the clot buster 108 in an unexpanded state. In FIG. 2, the clot buster 108 is a hollow cylinder 200 having a plurality of longitudinal cuts 124 dividing the surface of the cylinder 200 into a plurality of struts 122. The longitudinal cuts 124 do not extend to the face 202 of the cylinder 200 such that the surface of the cylinder 200 near a face 202 forms a complete circle.

The cylinder 200 may be expanded radially, moving each face 202 of the cylinder 200 towards one another. The struts 122 buckle extending outward at their midpoint. The cylinder 200 is then shape set so that the cylinder 200 is self-biased to the configuration shown in FIG. 1. For example, a cylinder may be expanded radially as described and then heat treated to remember the shape using known techniques.

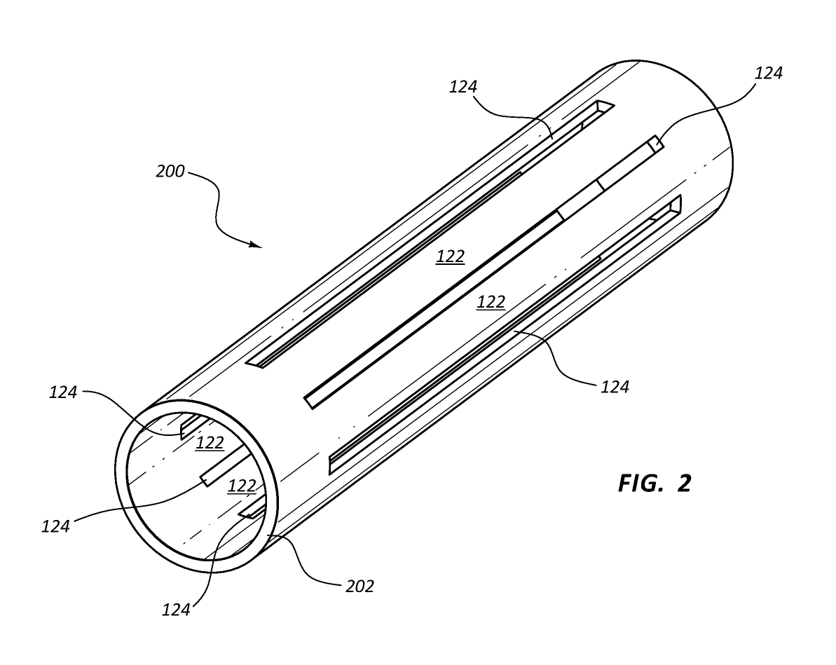

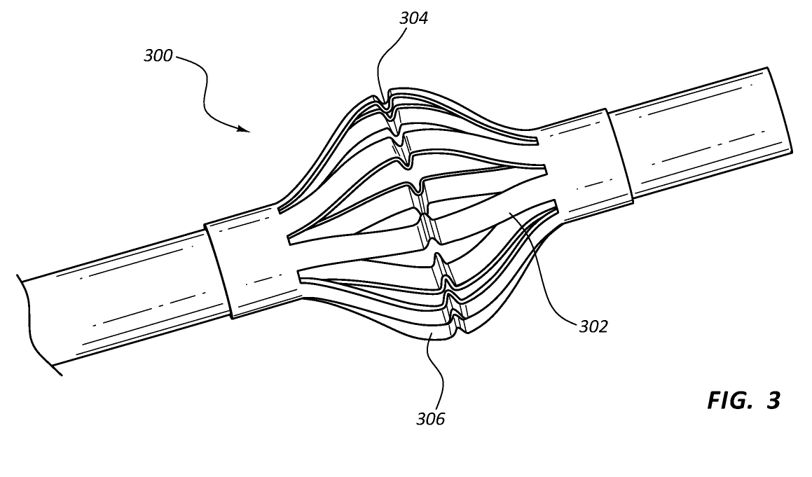

In some embodiments the clot buster may be formed in shapes other than the described double cone. For example, FIG. 3 through FIG. 5 illustrate alternative clot buster shapes. In FIG. 3, the clot buster 300 has blades 302 that have been shape set to have a dimple 304 disposed near a cone base 306. In FIG. 4, a clot buster 400 has blades 402 that have a more pronounced curve on the distal portion 408 as compared to the proximal portion. The distal portion 408 of the clot buster 400 has blades 402 that extend longitudinally before making at abrupt change at location 404 where the blades 402 extend outward, nearly perpendicular to the axis of the clot buster 400. Other angles are possible and the blades 402 do not need to extend perpendicularly to the axis.

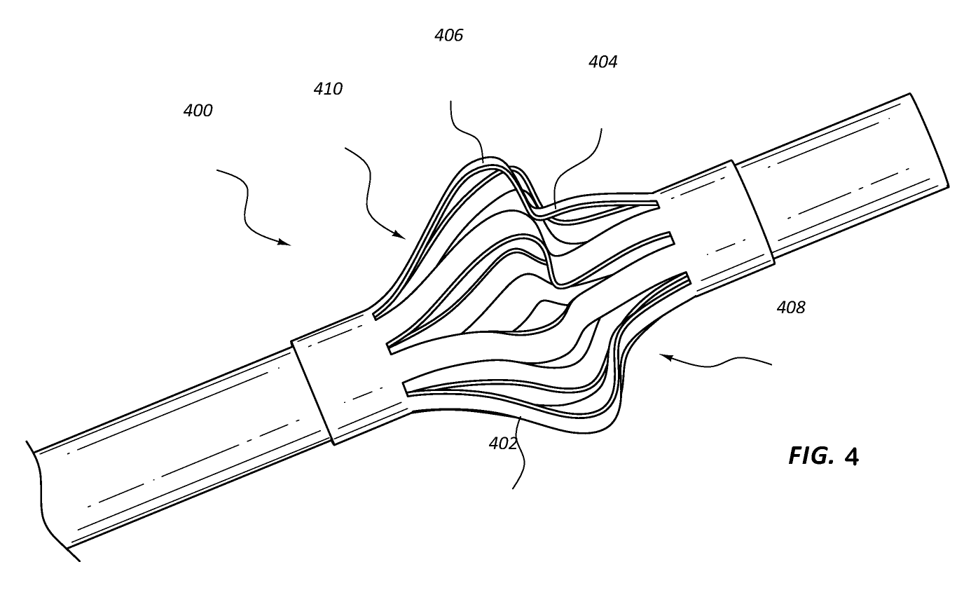

In FIG. 5, a clot buster 500 is shown in which the blades 502 are formed into different shapes. A first blade 504 is formed to have a zigzag 508 at a distal portion 512. A second blade 506 is formed to curve outward at the distal portion 512. Other combinations of blades are possible and are not limited by this disclosure. In some embodiments a clot buster may have every other blade removed from the base of the cone to the distal section of the clot buster.

Returning to FIG. 1, the first cone 110 section of the blades 122 has a coating applied to it that forms a web 124 between adjacent blades 122. When the clot buster 108 is expanded, as shown in FIG. 1, the web 124 forms a funnel 126 with an enlarged portion near the base 114 of the first cone 110 and a neck near the apex 118 of the first cone 118. The coating may be applied to the interior surface of the blades 122, to the exterior surface of the blades 122, or may be applied to both surfaces of the blades 122. In some embodiments, the coating may encapsulate the blades 122. The coating may be formed of an elastic polymer, allowing the coating to expand when the blades 122 are extended.

In the clot buster 108B embodied in FIG. 1b, a first cone 110B section of the blades 122B does not have any coating applied. Instead, a coating is applied to a second cone section 112B forming a web 124B between adjacent blades 122B in the second cone 112B section. When the clot buster 108B is expanded, as shown in FIG. 1B, the web 124B forms a basket 150B with an enlarged open portion 152B near the base 114 of the second cone 112B. In contrast to the embodiment of FIG. 1, the web 124B does not funnel material into the aspiration catheter, but may instead serve to catch clot particles that may flow distally from the clot buster 108B.

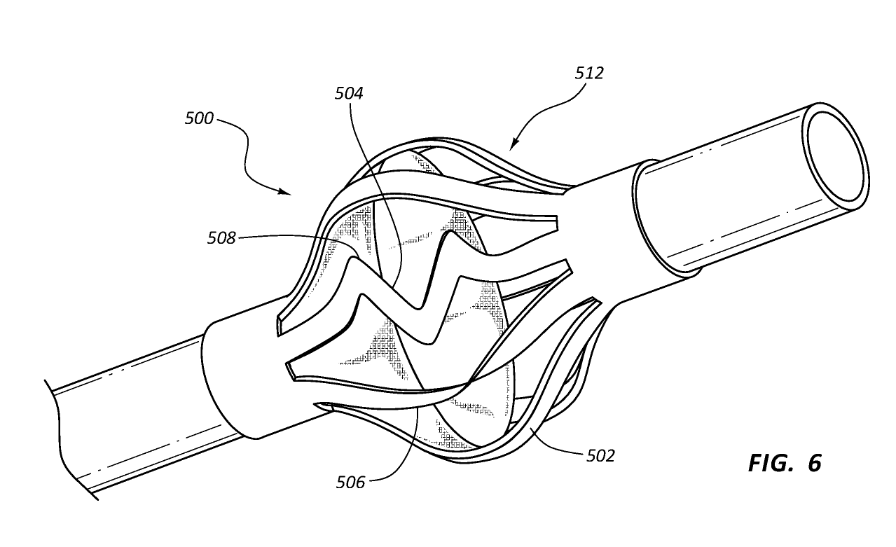

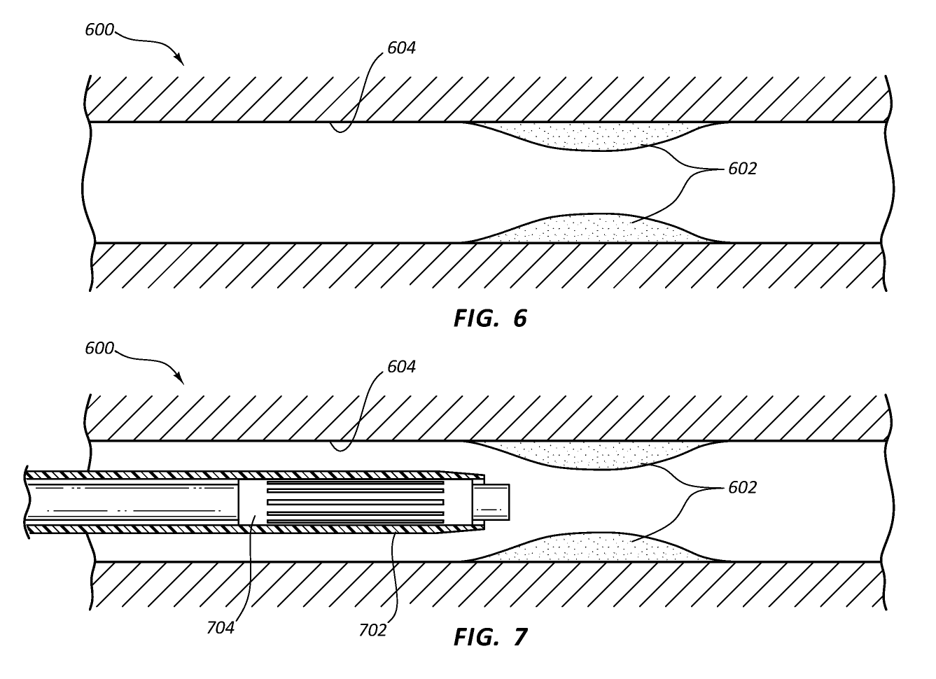

FIG. 6 illustrates a blood vessel 600 having a clot 602 formed on the wall 604 of the vessel 600. The clot 602 causes a restriction in the cross sectional area of the blood vessel 600 resulting in reduced blood flow and may potentially break free, traveling through the blood vessel 600 until it lodges in a vessel of reduced cross sectional area. A clot busting catheter may be guided to the clot 602 using commonly known techniques. In one embodiment, a surgeon may advance a wireguide to the clot 602 and a dilator catheter may then be advanced over the wire guide to the clot 602. A sheath may then be advanced over the dilator catheter to the clot 602. Once the sheath is in place, the dilator catheter may be removed, leaving the sheath. The clot busting catheter may then be easily advanced within the sheath.

In some embodiments a guidewire may be left in place and a clot busting catheter may be advanced over the guidewire. This may be done in within an in place sheath, or may be done independent of the sheath. In such embodiments, the clot buster catheter may have a lumen or other guide within the tubular body through which the guidewire extends. The tip of the clot buster may have a passage allowing the guidewire to pass through the tip.

FIG. 7 illustrates the blood vessel 600 of FIG. 6, but with a clot busting catheter 700 disposed within the vessel 600 proximate the clot 602. The clot busting catheter 700 has been guided to the clot 602 with a clot buster 704 in an unexpanded state, such that it can be easily guided to the clot 602. An introducer sheath 702 is disposed about the clot buster 704, constraining the clot buster 704 to its unexpanded state. The introducer sheath 702 is retractable, such that a surgeon may selectively retract the introducer sheath 702 freeing the clot buster 704 from the constraint posed by the introducer sheath 702. In some embodiments the introducer sheath 702 extends the length of the clot busting catheter 700 and may be guided to the clot 602 without the clot buster 704. The clot buster 704 may then be guided through the introducer sheath 702 to the site of the clot 602 in an unexpanded state. In such embodiments, when the clot buster 704 is advanced to the clot 602, the clot buster 704 may be extended from the sheath 702, freeing the clot buster 704 from the radial constraint of the sheath 702.

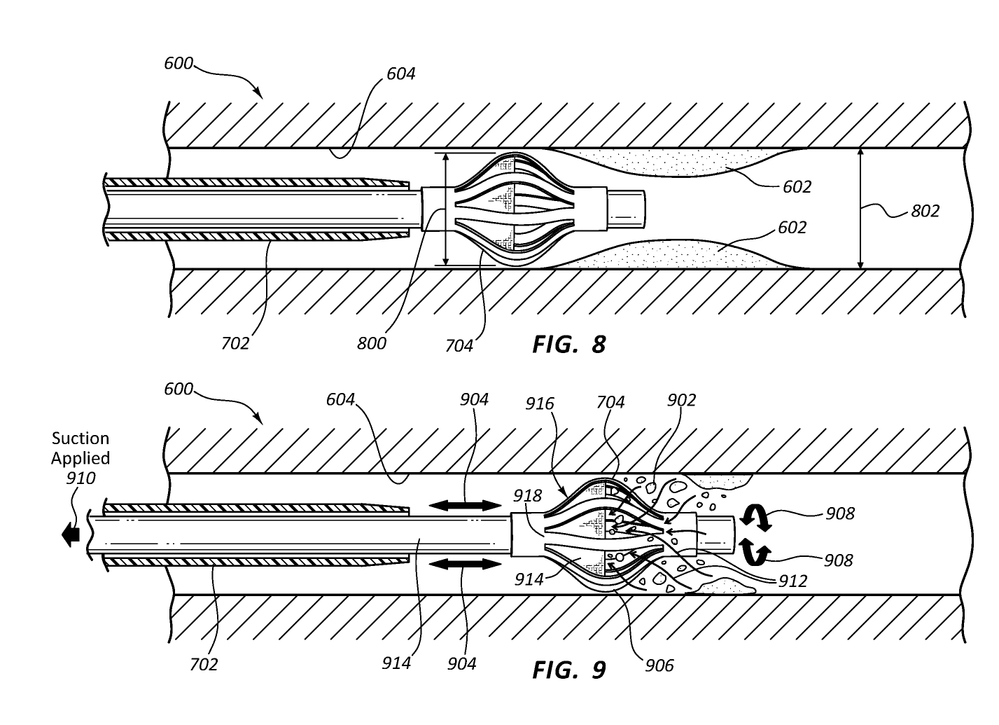

In FIG. 8 the introducer sheath 702 has been refracted proximally allowing the clot buster 704 to expand radially into its self-biased, expanded state. In other embodiments the clot buster 704 may be extended past the distal end of the introducer sheath 702 allowing the clot buster 704 to expand into its self-biased, expanded state. The clot buster 704 may be chosen such that a maximum outside diameter 800 of the expanded clot buster 704 is similar to an inside diameter 802 of the vessel proximate the clot 602. A distal tip 804 of the clot busting catheter 700 is guided to pass through the clot 602. In some embodiments, the distal tip 804 may serve as a dilator, expanding the clot 602 to enable to clot buster 704 to contact the clot 602.

FIG. 9 illustrates the clot buster 704 breaking up the clot 602 into clot pieces 902. The clot 602 may be broken into clot pieces 902 by moving the clot buster 704 axially as shown by arrow 904 such that open blades 906 engage the clot 602, and by spinning the clot buster 704 as shown by arrow 908 such that edges of the open blades 906 contact the clots 602. In some embodiments, the clot buster 704 may both be moved axially and spun to break up the clot 602.

A vacuum, indicated by arrow 910, is applied to a lumen 914 of the clot buster catheter 700 reducing the pressure in the lumen 914. Fluid, such as blood, proximate the clot buster 704 flows towards the reduced pressure and into the lumen 914 as indicated by arrows 912, carrying the clot pieces 902 along with it. Like the embodiment of FIG. 1, a coating 914 covers the proximal blades 906 to form a funnel 916. The funnel 916 channels the flow of fluid and clot pieces 902 into a neck 918 of the funnel 916 and into the lumen 914.

In embodiments having a guidewire, the clot buster 704 may spin about the guidewire or may move axially along the guidewire. Thus, the clot buster 704 may be moved proximally leaving the guidewire in place. The clot buster 704 may then be returned to the site of the clot 602 by advancing the clot buster 704 over the guidewire.

When the surgeon is finished breaking up the clot 602 and aspirating the pieces, the clot buster 704 may be retrieved in the reverse order that the clot buster 704 was deployed. The clot buster catheter 700 may be moved in a proximal direction away from the clot 602. The surgeon then advances the sheath 702 back over the clot buster 704, or retracts the clot buster 704 within the sheath 702. The sheath 702 once again provides a radial constraint to the clot buster 704, compressing the clot buster 704 into the unexpanded shape shown in FIG. 700. The clot buster catheter 700 may then be removed from the vessel.

In other embodiments, the coating may be applied to the distal portion of the blades and the proximal portion of the blades may be uncoated. In this embodiment the clot buster is used similar to the embodiment described previously, with the exception that the surgeon will advance the clot buster beyond the clot prior to retracting the sheath. Once deployed, the surgeon pulls the clot buster through the clot breaking up the clot into pieces. A vacuum is applied to the lumen and clot pieces and fluid is aspirated through the lumen. Clot pieces that are not aspirated are caught in the funnel. When the clot buster is retracted, the funnel envelops the clot pieces as the sheath compresses the clot buster. The clot buster catheter may then be removed by the surgeon.

Embodiments of the invention have been primarily described in relation to the double cone configuration of the clot buster, but the described methods may be used with other shapes of the clot buster, such as those shown in FIGS. 3, 4, and 5. It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

What is claimed:

1. A catheter assembly comprising:
a barrel formed from a shape memory material, the barrel having a first end having an outside diameter, a second end spaced apart from the first end, a longitudinal axis extending from the first end to the second end, a plurality of longitudinal blades extending proximate said first end to proximate said second end, and a film disposed on a portion of the plurality of blades, wherein the barrel is self-biased to an expanded state in which a portion of each of the longitudinal blades extends beyond the outside diameter of the first end;
an elongated tubular body having a lumen disposed therein and a distal end coupled to the first end of the barrel; and
a sheath disposed about the barrel, the sheath adapted to translate axially from a first position about the barrel to a second position about the elongated tubular body, wherein the first position constrains the barrel in a compacted configuration in which the plurality of longitudinal blades do not extend beyond the first outside diameter.

2. The catheter assembly of claim 1 further comprising an aspiration device adapted to lower a fluid pressure within the lumen of the elongated tubular body.

3. The catheter assembly of claim 1 wherein the film is disposed on an interior portion of the plurality of blades.

4. The catheter assembly of claim 1 wherein the film is disposed on an exterior portion of the plurality of blades.

5. The catheter assembly of claim 1 wherein the film envelopes a portion of the plurality of blades.

6. The catheter assembly of claim 1 wherein the film extends from the first end of the barrel to a mid-point of the barrel.

7. The catheter assembly of claim 1 wherein the film extends from the second end of the barrel to a mid-point of the barrel.

8. The catheter assembly of claim 1 further comprising at least one additional blade that extends only to a mid-point of the barrel.

9. The catheter assembly of claim 1 wherein at least one blade has a dimple near a mid-point of the barrel.

10. The catheter assembly of claim 1 wherein at least one blade is biased to extend longitudinally from the second end to proximate a midpoint of the barrel and extend radially from the midpoint.

11. The catheter assembly of claim 1 wherein at least one blade follows a zigzag pattern.

12. The catheter assembly of claim 1 wherein at least one blade is curved.

13. The catheter assembly of claim 1 wherein the first end of the barrel has an inner surface bonded to an outer surface of the elongated tubular body.

14. The catheter assembly of claim 1 wherein the first end of the barrel has an outer surface bonded to an inner surface of the elongated tubular body.

15. The catheter assembly of claim 1 further comprising a tip having a distal tip end and a proximal tip end, wherein the proximal tip end is joined to the second end of the barrel.

16. The catheter assembly of claim 1 wherein the distal tip end is tapered.

17. The catheter assembly of claim 1 wherein the film is impermeable.
